(19) United States
(12) Patent Application Publication
(10) Pub. No.: US 2007/0129661 A1
(43) Pub. Date: Jun. 7, 2007

(54) Anti-pronation device

(76) Inventor: Randolph W. Nordyke, Ventura, CA (US)

Correspondence Address: Phil Virga, #105, 1525 Aviation Blvd., Redondo Beach, CA, 90278, US

(21) Appl. No.: 11/295,955

(22) Filed: Dec. 6, 2005

(51) Int. Cl. A61F 13/06

(52) U.S. Cl. 602/66

(57) ABSTRACT

An anti-pronation device and method for supporting the longitudinal arch for stabilizing a foot against the forces of pronation are disclosed. The anti-pronation device is a specially shaped film that has adhesive on one side, which when strategically placed adheres to the rear foot just distal (in front of) to the heel of the foot. The anti-pronation device is designed to help realign the foot, in particular the rearfoot, which in turn helps to support the longitudinal arch of the foot.

### FIELD OF THE INVENTION

The invention relates generally to orthopedic devices for the foot and more specifically to supporting the longitudinal arch of the foot in a simple, more comfortable, easier to apply, cost-effective, unobtrusive manner.

### BACKGROUND

There are two anatomical arches of the foot, the transverse (metatarsal) arch and the longitudinal arch. The anti-pronation device described herein is concerned only with the longitudinal arch. Supporting this arch by mechanical means is an indicated and accepted modality for several conditions of the foot. General foot fatigue/excessive pronation and heel spur syndrome/plantar fasciitis are two of the more common conditions.

Pronation of the foot, although technically a motion involving all three anatomic planes, is essentially a rolling in of the foot and ankle, or in even more simple terms, a collapsing of the longitudinal arch. Pronation, along with its opposite counterpart, supination, is a regular and essential part of foot function when it occurs within a “normal” range. When excessive pronation occurs, however, lower extremity injuries usually result. To begin with, motion in joints that are not supposed to have much motion (hypermobility) cause muscles, and ligaments, which are not used to working during certain phases of gait or stance to become active in order to stabilize these joints. Most notably is excessive strain of the Plantar Fascia Ligament, also known as Plantar Fasciitis. Over a period of time additional pathology may result from this pronation syndrome.

It is well known in the orthopedic arts that the sub-talar joint, the joint between the talus and the calcaneus, is a key component to preventing excessive pronation of the foot and more specifically the rearfoot. In turn, if the rearfoot is pronated (everted), there is an “unlocking” mechanism that occurs to the midfoot (mid-tarsal joint), which causes the foot to “flatten out”, which subsequently puts additional load to the foot and to the supporting structure to the plantar aspect of the foot referred to as the plantar fascia.

If the foot is held in a neutral position, or a supinated (inverted) position, the alignment of the sub-talar joint does not allow for the unlocking mechanism of the mid-tarsal joint. The concept behind a successful orthotic device for the prevention of arch breakdown and for the prevention of plantar fasciitis is to prevent pronation of the rearfoot, which in turn keeps the mid-tarsal joint from unlocking.

Any orthotic or orthopedic device that can limit, resist, or stabilize this pronation tendency to any degree will allow its wearer to stand or walk in greater comfort and reduce the chances of further pathology. At present, the most widely accepted method of providing this support is by means of expensive molded arch support devices, commonly called custom orthotics. Therefore, a need exists to provide for an inexpensive over-the-counter device that reduces the pronation of the rearfoot while simultaneously helping to support the arch of the foot. Additionally, this device provides support and or reduces the pronation while allowing consumers the flexilbity of wearing open sandals or heels. The device may also be worn as a “test strip” applied by physicians trying to ascertain whether additional support to the foot would alleviate the patient's lower extremity symptoms. The foot device of the present invention solves these problems in a unique and novel fashion by utilizing the elongation principles of a specially shaped polyethylene adhesive film.

### SUMMARY

A new and simplified anti-pronation device and method for supporting the longitudinal arch for stabilizing a foot against the forces of pronation are disclosed. The anti-pronation device is a specially shaped film that has adhesive on one side, which when strategically placed adheres to the rear foot just distal (in front of) to the heel of the foot. The anti-pronation device is designed to help realign the foot, in particular the rearfoot, which in turn helps to support the longitudinal arch of the foot.

Other advantages and salient features of the invention will become apparent from the following detailed description, which, taken in conjunction with the annexed drawings, discloses the invention.

### BRIEF DESCRIPTION OF THE DRAWINGS

Referring now to the drawings that form a part of the original disclosure:

FIG. 1 is an outside plan view of the orthopedic foot device showing its general shape and dimensions;

FIG. 2 is a perspective view of the orthopedic foot device in an open and slightly curved configuration to show its general shape and dimensions;

FIGS. 3a through 3d illustrate one method for how the orthopedic foot device is applied to or wrapped around the foot; and

FIGS. 4a through 4d illustrate another method for how the orthopedic foot device is also applied to or wrapped around the foot.

The invention and its various embodiments may now be understood by turning to the following detailed description.

### DETAILED DESCRIPTION OF THE DRAWINGS

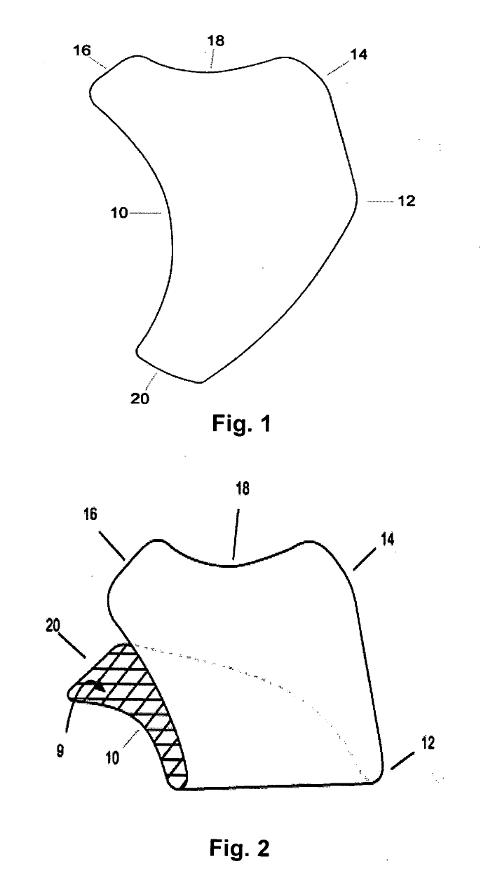

Referring now to FIGS. 1 and 2 an anti-pronation device 8 for the longitudinal arch of the foot is comprised of a flexible film material having an adhesive 9 disposed on at least one surface. The flexible film material 8 may consist of any natural or synthetic fibers such as from the group consisting of cotton, polyester, rayon, acrylic, polypropylene, nylon or polyethylene or blends thereof. By way of example only, the flexible film material 8 may be formed from a polyethylene material, that is relatively strong with enough flexibility to help create support for the foot, while being a comfortable and conforming material to the foot. The thickness of the flexible film material 8 is sufficiently thin to reduce any bulk to the plantar surface of the foot.

As shown in FIG. 1, the anti-pronation device is a specially designed, strategically placed, multi-sided strip of film 8 that has the adhesive 9 disposed on at least one surface for adhering to the rear foot just distal (in front of) to the heel of the foot. In use, the strip of film 8 is wrapped snugly around the longitudinal arch of the foot with the strip of film 8 strategically placed to provide optimum arch support and secured by adhesion to the skin of the foot, as will be more described below. By way of example, the adhesive 9 may be a hypoallergenic, pressure sensitive acrylate adhesive that provides superior adhesive strength while ensuring safe levels of adhesive for fragile skin.

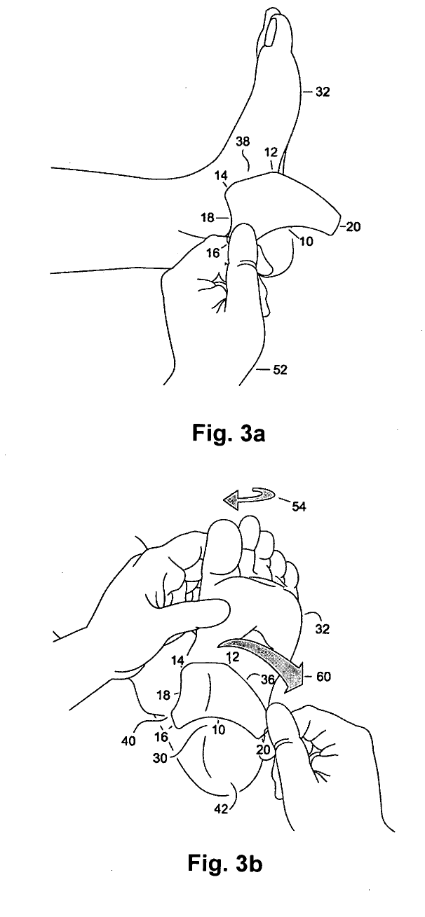

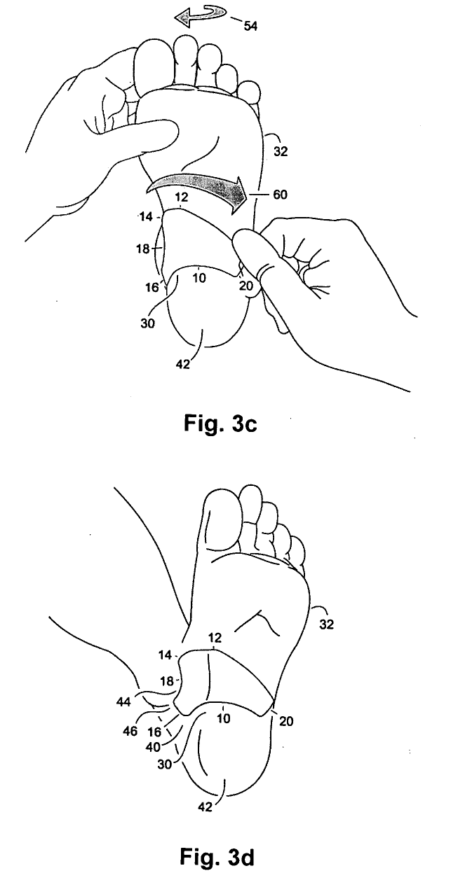

Turning once again to FIGS. 1 and 2, there is shown an anti-pronation device comprising an n-sided polygon shaped strip or flexible film wherein the dimensions and shape of the multi-sided strip of film 8 are defined by a hexagonally shaped strip having six (6) sides 10, 12, 14, 16, 18 and 20 respectively. It should be understood that any polygon shaped strip may be used as an anti-pronation device in accordance with the teachings of the present invention. As shown in FIG. 3a, the first curved side 10 of the hexagonally shaped strip of film 8 is such that it wraps just in front of the heel (distal heel) 30, wherein the various radii that define the first curved side 10 are chosen not to allow for any overall contact of the strip of film 8 with the ground or foot after placement. The design of the first curved side 10 results in the strip of film 8 staying adhered to foot 32 for a much longer period of time, and allows for greater comfort. More specifically, the first curved side 10 of the film 8 sits just distal to the inferior-distal aspect of the calcaneus 30 of a foot 32 as shown in FIG. 3a and 3b.

Referring once again to FIGS. 1 through 3, the most distal curved side 12 of film 8 is designed to allow for greater contact into the mid-arch 34 of foot 32, yet the overall width of the film 8 is not excessively wide so as to fit a high arched foot. After placement of the film 8 upon foot 32, the distal curved side 12 of the film 8 is located and sits under the inferior aspect of the mid-foot 36 and the Lis-francs joint of the foot 32 as shown in FIG. 3a. The sides 14 and 16 of the film 8 are referred to as first and second medial “arms” and are designed to provide additional surface area for the film 8 to assist in an “inversion pull” of the film 8 forcing the foot 32 to be placed into a more neutral position, or, a “less pronated” position, as will be more fully described below. The first medial “arm” or side 14 is located above the distal curved side 12 defining an upper aspect portion for the film 8 and is dimensioned and shaped to fit under and along the anterior aspect of the medial malleoli 40 as shown in FIG. 3c. More specifically, the first medial arm or side 14 sits on the medial aspect 38 of the talo-navicular joint of foot 32.

Turning once again to FIG. 3a, the second medial “arm” or side 16 defines a posterior aspect portion for film 8 and is dimensioned and shaped to allow for adhesion onto the medial aspect 40 of the heel 42, yet stops short of continuing to the most posterior aspect of heel 42 wherein testing has proved that when continued the film 8 became un-attached from the skin of the foot 32 too easily. More specifically, the second medial “arm” or side 16 of the film 8 sits on the posterior aspect of the medial malleoli 40, namely the tibia and the posterior aspect of the ankle mortice as shown in FIG. 3d.

Located between the first and second medial “arms” 14 and 16 is side 18 which defines an inferior portion of the arm of the film 8, and is shaped and designed to adhere below the midline of the medial malleoli 44 as shown in FIG. 3D. By fitting under the medial malleoli 44, the strip of film 8 is comfortable to the wearer, and does not cross or pull across a joint in the rearfoot or ankle 46, thus, the strip of film 8 stays in place longer for a more effective “pull” of the subtalar joint into a more neutral rear-foot position as shown in FIG. 3d.

Referring once again to FIGS. 1 through 3, the lateral “leg” or side 20 of the strip of film 8 is shaped and designed to wrap around the lateral-inferor aspect 48 of foot 32, namely, just lateral to the cuboid, and just proximal to the base of the 5^{th} metatarsal of foot 32. It should be noted and most importantly that the design of the strip of film 8 is such that it should “end” on the lateral plantar edge 50 of the foot 32 as described above and shown in FIGS. 3c and 3d, and NOT wrap up on the lateral edge of the foot 32. Thus, by not going up the lateral aspect of the foot, the foot has a much better chance of inverting, and NOT being in a pronated (everted) position.

With the six (6) sides of the strip of film 8 now having been described, FIGS. 3a through 3d illustrate one method for how the strip of film 8 is applied to and wrapped around the foot 32. First, as shown in FIG. 3a, sides 14, 16 and 18 of the strip of film 8 is adhered to the medial aspect of foot 32 by locating and applying the adhesive surface 9 under the medial malleoli 38 (inside ankle bone) of the foot 32. The “leg” of the strip 20 of film 8 is then grasped by hand 52. Next, as shown in FIG. 3b, the foot 32 is then inverted (supinated) 54, and the strip of film 8 is then applied under the bottom of the foot 32, just in front of the heel 42, and towards the lateral plantar aspect of the foot 32 as shown in FIG. 3c. Lastly and most Importantly, the strip of film 8 is designed to stop at the plantar lateral edge of the foot 32, and does NOT get applied up the lateral side of the foot.

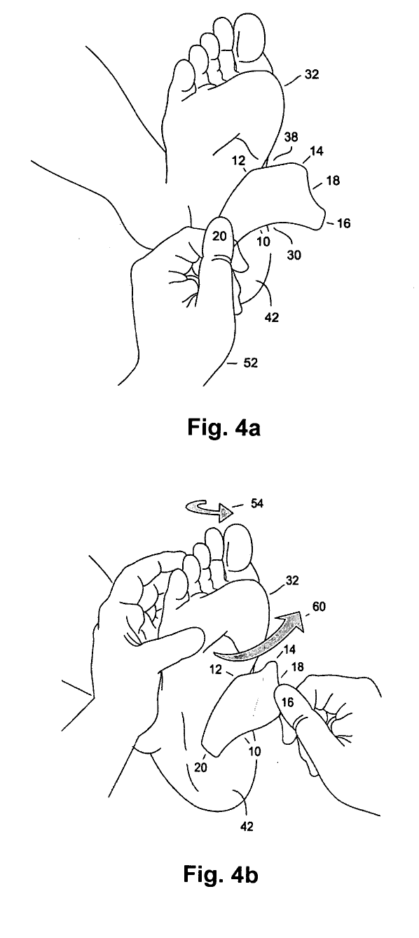

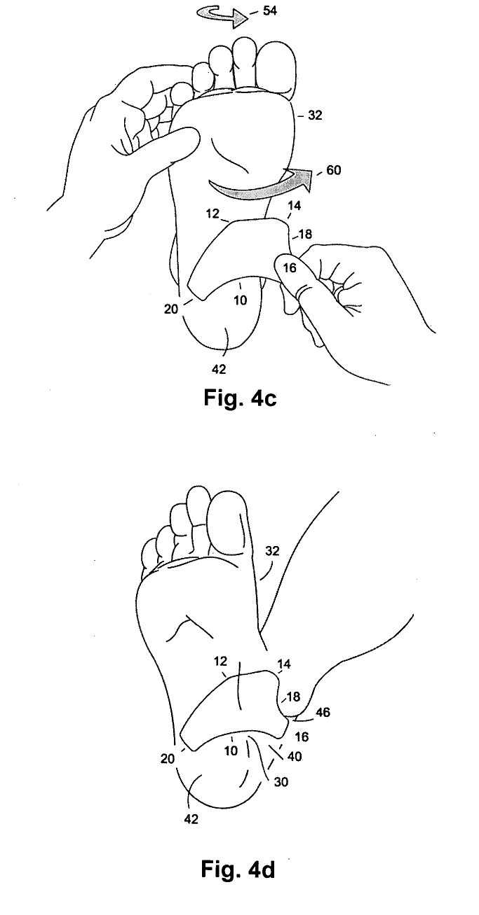

FIGS. 4a through 4d illustrate another method for how the orthopedic foot device is also applied to or wrapped around the foot. As shown in FIG. 4a, the foot device 8 is adhered to the foot by applying it to the lateral side 20 and grasping by hand 52 portions 14, 16 and 18 of the strip's sides first, then inverting the foot once again as shown in FIG. 4b, and bringing it under the medial malleolus 38. By having the foot in an inverted (supinated) 54 position, with the strip of film wrapping up the medial side of the foot, and NOT up the lateral side of the foot 32, the foot 32 is held in a more neutral position, which prevents the unlocking mechanism of the mid-tarsal joint. Although not shown, the strip of film 8 may be perforated throughout to allow the foot to breath providing more comfort to the consumer.

Both of these methods of application keep the sub-talar joint in a more inverted position, which is paramount for the anti-pronation device 8 being effective. Additionally, the anti-pronation device 8 is designed as a disposable product that a consumer or patient may wear for 24 hours. By way of example but not of limitation, the strip of film 8 may be a single coated tape consisting of a matte finish, 5 mil transparent polyethylene, coated with a hypoallergenic, pressure sensitive acrylate adhesive. Additionally, the consumer may purchase the film having a removable liner wherein the liner is a silicone treated, polyethylene coated one side only, bleached Kraft paper. The liner is also used for storage and transport purposes. It should also be noted that there are no adverse effects that occur as a result of prolonged wear beyond 24 hours, however the benefits begin to diminish after a period of time.

While advantageous embodiments have been chosen to illustrate the invention, it will be understood by those skilled in the art that various changes and modifications may be made therein without departing from the scope of the invention as defined in the appended claims.

What is claimed is:

1. An anti-pronation device for reducing pronation of a rearfoot of a foot and heel, comprising:
a flexible film defined by a plurality of sides having an adhesive disposed on at least one surface and when the foot is placed in an inverted position said flexible film is placed and adhered to the foot wherein said plurality of sides are shaped and located to reduce pronation of the foot.

2. The anti-pronation device according to claim 1, wherein said plurality of sides of said flexible film further define:
a first curved side shaped to allow a portion of said flexible film to be located in front of the heel of the foot wherein placement of said flexible film onto the foot does not allow said flexible film to make contact with ground during use.

3. The anti-pronation device according to claim 1, wherein said plurality of sides of said flexible film further define:
a distal curved side wherein said distal curved side is shaped to allow a portion of said flexible film to be located under the inferior aspect of the mid-foot portion of the foot.

4. The anti-pronation device according to claim 1, wherein said plurality of sides of said flexible film further define:
a first medial arm shaped to provide a sufficient surface area of said flexible film for allowing an inversion pull of said flexible film for forcing the foot into a neutral position during application.

5. The anti-pronation device according to claim 1, wherein said plurality of sides of said flexible film further define:
a second medial arm shaped to allow a portion of said flexible film to be adhered to a medial aspect of the heel of the foot and stop short of continuing to the posterior aspect of the heel.

6. The anti-pronation device according to claim 1, wherein said plurality of sides of said flexible film further define:
A lateral side shaped to allow a portion of said film to be located inside an ankle bone of the foot and ending at a lateral plantar edge of the foot.

7. The anti-pronation device according to claim 1, wherein said flexible film further defines perforations.

8. The anti-pronation device according to claim 1, wherein said plurality of sides of said flexible film further defines six sides.

9. The anti-pronation device according to claim 1, wherein said flexible film attached to a removable liner for storage and transport before use.

10. An anti-pronation strip for supporting the longitudinal arch for stabilizing the foot against the forces of pronation comprising:
an n-sided polygon shaped strip having adhesive means on one side whereby said n-sided polygon shaped strip adheres to the rear foot in front of the heel of the foot thereby realigning the rearfoot which in turn helps to support the longitudinal arch of the foot.

11. The anti-pronation device according to claim 10, wherein said n-sided polygon shaped strip further defines:
a first curved side shaped to allow a portion of said n-sided polygon shaped strip to be located in front of the heel of the foot wherein placement of said n-sided polygon shaped strip onto the foot does not allow said flexible film to make contact with ground during use.

12. The anti-pronation device according to claim 10, wherein said n-sided polygon shaped strip further defines:
a distal curved side wherein said distal curved side is shaped to allow a portion of said n-sided polygon shaped strip to be located under the inferior aspect of the mid-foot portion of the foot.

13. The anti-pronation device according to claim 10, wherein said n-sided polygon shaped strip further defines:
a first medial arm shaped to provide a sufficient surface area of said n-sided polygon shaped strip for allowing an inversion pull of said n-sided polygon shaped strip for forcing the foot into a neutral position during application.

14. The anti-pronation device according to claim 10, wherein said n-sided polygon shaped strip further defines:
a second medial arm shaped to allow a portion of said n-sided polygon shaped strip to be adhered to a medial aspect of the heel of the foot and stop short of continuing to the posterior aspect of the heel.

15. The anti-pronation device according to claim 10, wherein said n-sided polygon shaped strip further defines:
a lateral side shaped to allow a portion of said n-sided polygon shaped strip to be located inside an ankle bone of the foot and ending at a lateral plantar edge of the foot.

16. The anti-pronation device according to claim 10, wherein said n-sided polygon shaped strip further defines perforations.

17. The anti-pronation device according to claim 10, wherein said n-sided polygon shaped strip further defines six sides.

18. An anti-pronation device for reducing pronation of a rearfoot of a foot and heel, comprising:
a flexible film defined by a plurality of sides having an adhesive disposed on at least one surface and when the foot is placed in an inverted position said flexible film is placed and adhered to the foot wherein said plurality of sides are shaped and located to reduce pronation of the foot;
a first curved side shaped to allow a portion of said flexible film to be located in front of the heel of the foot wherein placement of said flexible film onto the foot does not allow said flexible film to make contact with ground during use;
a distal curved side wherein said distal curved side is shaped to allow a portion of said flexible film to be located under the inferior aspect of the mid-foot portion of the foot;
a first medial arm shaped to provide a sufficient surface area of said flexible film for allowing an inversion pull of said flexible film for forcing the foot into a neutral position during application;
a second medial arm shaped to allow a portion of said flexible film to be adhered to a medial aspect of the heel of the foot and stop short of continuing to the posterior aspect of the heel; and
a lateral side shaped to allow a portion of said film to be located inside an ankle bone of the foot and ending at a lateral plantar edge of the foot.

19. The anti-pronation device according to claim 1, wherein said flexible film further defines perforations.

20. The anti-pronation device according to claim 1, wherein said plurality of sides of said flexible film further defines six sides.
